SEQUENCE LISTING (15 sequences)

SEQ ID NO: 1 - protein, 13 aa, Gallus gallus
LGESADLTAISVR

SEQ ID NO: 2 - protein, 13 aa, Gallus gallus
AVQLGLGPFPYCR

SEQ ID NO: 3 - protein, 8 aa, Gallus gallus
DVVDLAQK

SEQ ID NO: 4 - protein, 12 aa, Gallus gallus
EPDNFWVALGGK

SEQ ID NO: 5 - protein, 17 aa, Gallus gallus
HTQVQVLPESGETPLFK

SEQ ID NO: 6 - protein, 15 aa, Gallus gallus
IEKVPFDAATLHTSR

SEQ ID NO: 7 - protein, 14 aa, Gallus gallus
LAAAVSNFGYDLYR

SEQ ID NO: 8 - protein, 8 aa, Gallus gallus
LLEGEESR

SEQ ID NO: 9 - protein, 12 aa, Gallus gallus
LQSLFTSPDFTK

SEQ ID NO: 10 - protein, 13 aa, Gallus gallus
LTPVAQELKELTK

SEQ ID NO: 11 - protein, 15 aa, Gallus gallus
NSDNDLVVNGIEIYR

SEQ ID NO: 12 - protein, 13 aa, Gallus gallus
QEYDESGPSIVHR

SEQ ID NO: 13 - protein, 9 aa, Gallus gallus
VELQELNDR

SEQ ID NO: 14 - protein, 9 aa, Gallus gallus
VSAYIPWIK

SEQ ID NO: 15 - protein, 8 aa, Gallus gallus
VTVASWMR

(19) United States
(12) Patent Application Publication
(10) Pub. No.: US 2026/0151437 A1
(43) Pub. Date: Jun. 4, 2026

(54) MYOGENIC COMPOUNDS

(71) Applicant: Myos Corp., Cedar Knolls, NJ (US)

(72) Inventors: Joseph MANNELLO, Cedar Knolls, NJ (US); Neerav D. PADLIYA, Cedar Knolls, NJ (US); Maghsoud DARIANI, Cedar Knolls, NJ (US)

(21) Appl. No.: 19/119,493

(22) PCT Filed: Oct. 18, 2023
(86) PCT No.: PCT/US2023/077141

§ 371 Date: Apr. 9, 2025

Related U.S. Application Data
(60) Provisional application No. 63/417,479, filed on Oct. 19, 2022.
(60) Provisional application No. 63/433,821, filed on Dec. 20, 2022.
(60) Provisional application No. 63/503,027, filed on May 18, 2023.

(51) Int. Cl. A61K 35/57; A61K 38/17; A61P 21/06

(52) U.S. Cl. CPC A61K 35/57; A61K 38/1703; A61P 21/06

(57) ABSTRACT

Myogenic compounds identified in egg yolk and synthetic derivatives thereof, nutraceutical compositions of one or more of these myogenic compounds and synthetic derivatives thereof and use of these compounds and nutraceutical compositions in increasing muscle mass in mammals are provided.

This patent application claims the benefit of priority from U.S. Provisional Application Ser. No. 63/417,479, filed Oct. 19, 2022, U.S. Provisional Application Ser. No. 63/433,821, filed Dec. 20, 2022 and U.S. Provisional Application Ser. No. 63/503,027, filed May 18, 2023, teachings of each of which are incorporated herein by reference in their entireties.

### FIELD

This disclosure relates to myogenic compounds identified in separated fractions of fertilized egg yolk and use of these myogenic compounds and combinations thereof or synthetic derivatives and combinations thereof in increasing muscle mass or decreasing muscle loss in mammals.

### BACKGROUND

There is a general interest in increasing muscle mass among professional athletes as well as the general population. In addition, sarcopenia or muscle wasting is a problem among the elderly affecting 5-13% percent of those aged 60-70 and 15-50% above the age of 80 (Shafiee et al. Journal of Diabetes & Metabolic Disorders, 2017 16(1)).

Previous studies have identified myogenic benefits from adding protein supplement derived from fertilized hen egg yolk extract to the diet (Sharp et al. Journal of the American College of Nutrition, 2016 35(8): 679-691; Evans et al. The Journals of Gerontology: Series A, 2020 76(1):108-114).

Fertilized egg yolk has been used in the past as a health food, specifically in Asian cultures, as a food called Balut. Additionally, studies in canines have shown that feed supplemented with fertilized egg yolk product, FORTETROPIN, has improved the outcome of tibial plateau leveling osteotomy by reducing muscle atrophy in the affected limb (White et al. PLoS ONE 2020 15(4): p. 1-10).

Egg yolk is made up of 70% lipids and 30% protein by dry weight with the lipids being triglycerides, phospholipids, and cholesterols and the proteins being low-density lipoproteins (LDL), high density lipoproteins (HDL), phosvitin, livetin, and riboflavin-binding protein (Mann, K. & Mann, M. PROTEOMICS 2008 8(1):178-191).

Due to its wide consumption, egg yolk has been extensively studied. As early as 1949, Romanoff et al. described the separation of plasma and granule portions of the egg (The avian egg 1949 New York: J. Wiley). Those experiments, as well as additional studies in 1989 by Burley and Vadehra (The avian egg: chemistry and biology. 1989, New York: Wiley), describe the structure, biology, chemistry, and development of the egg yolk. It was found that the yolk can easily be separated into a plasma and granule fractions using centrifugation (McBee, L. & Cotterill, 0. Poultry Science 1973 Oxford Univ Press Gret Clarendon St. Oxford OX2 6DP, England) with the plasma and granule portions containing equal amounts of protein and 90%:10% of the lipids, respectively. The plasma contains 85% LDL and 15% livetin. The insoluble granules are mainly composed of phosvitin and HDL linked by association with calcium molecules.

It is currently unknown what compounds present within fertilized egg yolk produce the myogenic effects observed.

### SUMMARY

This disclosure relates to myogenic compounds identified in a separated fraction of fertilized avian egg yolk.

An aspect of this disclosure relates to compositions comprising one or more of these myogenic compounds or synthetic derivatives thereof.

In one nonlimiting embodiment, the composition comprises one or more myogenic compounds identified in subfraction F7.11, F7.18, F7.21, FS.10, FS.11, and/or FS.22 of fertilized avian egg yolk or a synthetic derivative thereof.

In one nonlimiting embodiment, the composition comprises one or more myogenic compounds comprising one or more peptide sequences depicted in Table 2 or a myogenically active fragment thereof.

In one nonlimiting embodiment, the myogenic compound is a protein selected from gelsolin, actin-depolymerizing factor, vimentin, SERPIN domain-containing protein, also referred to as pigment epithelium derived factor (PEDF), hepatocyte growth factor activator, chick nucleoside diphosphate kinase, inter-alpha-trypsin inhibitor heavy chain, keratin-type II cytoskeletal cochleal, desmin, apolipoprotein A-I, albumin, actin-cytoplasmic type 5, actin-cytoplasmic 1, vitellogenin-1, actin-cytoplasmic 2, IF rod domain-containing protein, vitellogenin-3, glial fibrillary acidic protein, plasminogen or type II alpha-keratin IIA or a myogenically active peptide fragment thereof.

In one nonlimiting embodiment, the myogenic compound is a protein selected from albumin, ovalbumin, gelsolin, lysozyme C, SMB domain-containing protein, transthyretin, IG-like domain-containing protein, fibrinogen C-terminal domain-containing protein, plasminogen, phosvitin (VTG2), peptidase S1 domain-containing protein or fibrinogen C or a myogenically active fragment thereof.

In one nonlimiting embodiment, the myogenic compound is a peptide sequence or protein which exhibits at least 70%, 80%, 90%, 95% or 99% sequence identity to a peptide sequence or protein identified herein and exhibits myogenic activity.

In one nonlimiting embodiment, the composition comprises one or more synthetic derivatives of a myogenic compound identified in subfraction F7.11, F7.18, F7.21, FS.10, FS.11, and/or FS.22 of fertilized avian egg yolk or comprising one or more peptide sequences depicted in Table 2.

In one nonlimiting embodiment, the myogenic compound is a protein or myogenically active peptide fragment thereof similar to that identified herein but is from a species alternative to chick such as, but not limited to, humans, dogs, cats, horses, cows, sheep, pigs and primates.

Another aspect of this disclosure relates to nutraceutical and/or pharmaceutical compositions comprising one or more myogenic compounds identified in a separated fraction of fertilized avian egg yolk or synthetic derivatives thereof and one or more nutraceutically and/or pharmaceutically acceptable excipients.

In one nonlimiting embodiment, the nutraceutical and/or pharmaceutical composition comprises one or more myogenic compounds identified in subfraction F7.11, F7.18, F7.21, FS.10, FS.11, and/or FS.22 of fertilized avian egg yolk or a synthetic derivative thereof.

In one nonlimiting embodiment, the nutraceutical and/or pharmaceutical composition comprises one or more myogenic compounds comprising one or more peptide sequences depicted in Table 2 or a myogenically active fragment thereof.

In one nonlimiting embodiment, the myogenic compound is a protein selected from gelsolin, actin-depolymerizing factor, vimentin, SERPIN domain-containing protein, also referred to as pigment epithelium derived factor (PEDF), hepatocyte growth factor activator, chick nucleoside diphosphate kinase, inter-alpha-trypsin inhibitor heavy chain, keratin-type II cytoskeletal cochleal, desmin, apolipoprotein A-I, albumin, actin-cytoplasmic type 5, actin-cytoplasmic 1, vitellogenin-1, actin-cytoplasmic 2, IF rod domain-containing protein, vitellogenin-3, glial fibrillary acidic protein, plasminogen or type II alpha-keratin IIA or a myogenically active peptide fragment thereof.

In one nonlimiting embodiment, the myogenic compound is a protein selected from albumin, ovalbumin, gelsolin, lysozyme C, SMB domain-containing protein, transthyretin, IG-like domain-containing protein, fibrinogen C-terminal domain-containing protein, plasminogen, phosvitin (VTG2), peptidase S1 domain-containing protein or fibrinogen C or a myogenically active fragment thereof.

In one nonlimiting embodiment, the myogenic compound is a peptide sequence or protein which exhibits at least 70%, 80%, 90%, 95% or 99% sequence identity to a peptide sequence or protein identified herein and exhibits myogenic activity.

In one nonlimiting embodiment, the composition comprises one or more synthetic derivatives of a myogenic compound identified in subfraction F7.11, F7.18, F7.21, FS.10, FS.11, and/or FS.22 of fertilized avian egg yolk or comprising one or more peptide sequences depicted in Table 2.

In one nonlimiting embodiment, the myogenic compound is a protein or myogenically active peptide fragment thereof similar to that identified herein but is from a species alternative to chick such as, but not limited to, humans, dogs, cats, horses, cows, sheep, pigs and primates.

Another aspect of this disclosure relates to a method for increasing muscle mass in a mammal which comprises administering to the mammal a composition comprising one or more myogenic compounds identified in a subfraction of fertilized avian egg yolk or a synthetic derivative thereof.

In one nonlimiting embodiment, the composition comprises one or more myogenic compounds identified in subfraction F7.11, F7.18, F7.21, FS.10, FS.11, and/or FS.22 of fertilized avian egg yolk or a synthetic derivative thereof.

In one nonlimiting embodiment, the composition comprises one or more myogenic compounds comprising one or more peptide sequences depicted in Table 2 or a myogenically active fragment thereof.

In one nonlimiting embodiment, the myogenic compound is a protein selected from gelsolin, actin-depolymerizing factor, vimentin, SERPIN domain-containing protein, also referred to as pigment epithelium derived factor (PEDF), hepatocyte growth factor activator, chick nucleoside diphosphate kinase, inter-alpha-trypsin inhibitor heavy chain, keratin-type II cytoskeletal cochleal, desmin, apolipoprotein A-I, albumin, actin-cytoplasmic type 5, actin-cytoplasmic 1, vitellogenin-1, actin-cytoplasmic 2, IF rod domain-containing protein, vitellogenin-3, glial fibrillary acidic protein, plasminogen or type II alpha-keratin IIA or a myogenically active peptide fragment thereof.

In one nonlimiting embodiment, the myogenic compound is a protein selected from albumin, ovalbumin, gelsolin, lysozyme C, SMB domain-containing protein, transthyretin, IG-like domain-containing protein, fibrinogen C-terminal domain-containing protein, plasminogen, phosvitin (VTG2), peptidase S1 domain-containing protein or fibrinogen C or a myogenically active fragment thereof.

In one nonlimiting embodiment, the myogenic compound is a peptide sequence or protein which exhibits at least 70%, 80%, 90%, 95% or 99% sequence identity to a peptide sequence or protein identified herein and exhibits myogenic activity.

In one nonlimiting embodiment, the composition comprises one or more synthetic derivatives of a myogenic compound identified in subfraction F7.11, F7.18, F7.21, FS.10, FS.11, and/or FS.22 of fertilized avian egg yolk or comprising one or more peptide sequences depicted in Table 2.

In one nonlimiting embodiment, the myogenic compound is a protein or myogenically active peptide fragment thereof similar to that identified herein but is from a species alternative to chick such as, but not limited to, humans, dogs, cats, horses, cows, sheep, pigs and primates.

### DETAILED DESCRIPTION

Provided by this disclosure are myogenic compounds identified in subfractions of fertilized avian egg yolk and synthetic derivatives thereof as well as nutraceutical compositions and/or pharmaceutical compositions comprising one or more of the myogenic compounds or synthetic derivatives thereof and methods for use of these compositions in increasing muscle mass in a mammal.

By “myogenic” or “myogenically active” as used herein it is meant a compound which increases muscle growth and/or decreases muscle loss. In one nonlimiting embodiment, the myogenic compound increases myogenic differentiation in a cell line with a reporter gene that is expressed under the promotion of myogenic transcription factors. In one nonlimiting embodiment, the myogenic compound increases muscle growth in a mammal. Increases are determined by comparison to differentiation and/or muscle growth in the absence of the myogenic compound and/or upon administration of a negative control. In one nonlimiting embodiment, the myogenic compound of the disclosure increases muscle growth in a mammal similarly to powdered egg yolk. In one nonlimiting embodiment, the myogenic compound of this disclosure increases muscle growth to a greater extent as compared to powdered egg yolk. In one nonlimiting embodiment, the myogenic compound of the disclosure decreases muscle loss in a mammal. In one nonlimiting embodiment, the myogenic compound of the disclosure decreases muscle loss in a mammal similarly to powdered egg yolk. In one nonlimiting embodiment, the myogenic compound of this disclosure decreases muscle loss to a greater extent as compared to powdered egg yolk.

In one nonlimiting embodiment, the myogenic compound upregulates mTor pathway activity, downregulates ubiquitin proteasome pathway activity, downregulates serum myostatin levels and/or reduces ActRIIB expression in the mammal.

By “mammal” as used herein, it is meant to include, but is not limited to, humans, dogs, cats, horses, cows, sheep, pigs and primates.

Myogenic compounds of this disclosure are distinguishable from natural egg yolk in that the myogenic compounds are either physically separated by mechanical means from other components in natural egg yolk including, but not limited to, non-myogenic components of egg yolk and lipid components of egg yolk, or produced by synthetic means, for example, via peptide synthesis or recombinant protein production by well-established protocols. Further, as will be understood by the skilled artisan upon reading this disclosure, while identified in fertilized avian egg yolk, other alternative biological sources are expected to contain the identified myogenic compounds and myogenic compounds of this disclosure may be derived from such alternative biological sources.

Skeletal muscle is a complex tissue composed mainly of myofibers that come in 4 different forms based on their myosin heavy chain: type I, type IIa, type IIx, and type IIb (Schiaffino, S. & Reggiani, C. Physiological Reviews 2011 91(4):1447-1531). Slow twitch fibers are mainly composed of type I while fast twitch fibers are composed of all forms of type II. Type I fibers are responsible for continuous use and perform postural functions while type II fibers are involved in tasks such as walking or running. Although both fiber types are constantly maintained and can be repaired, it is well documented that sarcopenia results in a deficit of type 2 fibers (Brunner et al. J Aging Phys Act 2007 15(3): 336-48).

A cell line with a reporter gene that is expressed under the promotion of myogenic transcription factors was used to identify myogenic differentiation resulting from various fractions of egg yolk separated into lipid and protein constituents. Using the MLC2 promoter region, a reporter cell line was developed from C2C12 myoblasts whose luciferase expression was correlated with a myogenic phenotype. C2C12 myoblasts were selected as they are relevant to muscle differentiation. The promoter region is known to be associated with adult muscle growth. More specifically, the MLC1f promoter was selected to drive expression of the luciferase transgene as it is expressed only in type IIb fibers in the rat (Neville Dev Genet 1996 19(2):157-62) and therefore is useful gene for identifying myoblast fusion and myofiber maturation as it relates to muscle differentiation.

A Mus musculus C2C12 myoblast cell line which can be induced to differentiate, forming myotubes was selected for this cell line. These cells were derived from normal CH3 mice that underwent a crush injury 2 days prior to isolation to produce satellite cells (Yaffe, D. & Saxel, O. R. A. Nature (London), 1977. 270(5639):725-727). These cells were further expanded and a subclone that is diploid and reliably forms myotubes was selected (Blau et al. Science (American Association for the Advancement of Science), 1985 230(4727):758-766). These cells were transfected using a lentivirus containing a plasmid composed of three parts, the MLC1f promoter (Neville et al. Dev Genet, 1996. 19(2):157-62), a blasticidin resistance gene, and the GLuc gene (New England BioLabs, Ipswich MA), which expresses Gaussia Luciferase, an enzyme that is not expressed in mammalian cell and which reacts with coelenterazine to produce luminescence.

The C2C12 cell line was transfected with this lentiviral vector and then selected using blasticidin supplemented media. After clonal expansion of the blasticidin resistant cells, the efficacy of the reporter was confirmed by assaying the differentiation of the cells. The cell line was then tested using three groups: a positive control containing 50 ng/ml of insulin-like growth factor (IGF) 1, a negative control containing 40 μg/ml of Dexamethasone, and a media control. Luciferase expression was measured using the Biolux Gaussia Luciferase assay kit and correlated with gene expression, and image analysis of fluorescently stained cells.

Egg yolk was then separated into protein and lipid fractions. Two different methods for separating proteins and lipids in egg yolk were used. The first method involved centrifugation and alginate to create three different fractions containing different concentrations of protein, fat and cholesterol. The second method removed lipid from the egg yolk with organic solvents.

Fertilized eggs were processed to remove the shells and vitellin membrane leaving the raw yolk. Raw yolk was then diluted with ddH₂O at a 2:3 ratio (v/v, egg yolk:ddH₂O) and pH was adjusted to 7. Egg yolk was kept overnight in a cold room (4° C.) on a stir plate set at a low speed (60 rpm). Next the yolk was centrifuged at 10,000 g and 4° C. for 45 minutes to produce a pellet of the granule fraction and a supernatant containing soluble proteins and lipid. Sodium alginate stock solution 1% (w/v) was then added to the supernatant at a ratio of 1:9 to produce 0.1% w/v sodium alginate in the final solution. The resulting solution was then centrifuged at 20° C. and 10,000 g for 15 minute to produce an aqueous fraction and an alginate-lipidic paste (Lipid). These fractions: granule, lipidic paste, and aqueous were then analyzed to determine their general composition. Cell studies were also performed using the fractions as media supplements to determine their effect on the myogenic differentiation of myoblasts in vitro.

SDS PAGE was used to determine the composition of each fraction based upon protein size. Samples were obtained from each fraction and mixed with 2× Laemmli Buffer containing 5% (v/v) β-mercaptoethanol. Samples were then warmed to 70° C. using a water bath for 15 minutes and loaded into lanes in duplicate using premade polyacrylamide gels mini-protean gtx. Gels were run at 200v for approximately 30 minutes. Running of the gels was stopped when the dye front reached the black reference line on the gel. Results were compared to those previously disclosed by Laca et al. (Food Hydrocolloids—FOOD HYDROCOLLOID 2010 24:434-443) for unfertilized eggs and showed similar results thus indicating that fertilization of egg yolks does not caused a significant change in the composition of the yolk and that myogenic compounds can also be derived from unfertilized egg.

For cell studies, differentiation was measured using the luciferase assay on cell media collected at various time points through the assay. Metabolic activity of each fraction group at each time point was also measured using a PrestoBlue assay to provide information on the biocompatibility of the fractions added to the media and to ensure that the addition of the fractions did not have a negative effect on the growth and fusion of cells. On days 0, 3, 7 and 10, media was removed from each of the fraction groups, frozen for later analysis, and then replaced with media containing 1:10% v/v PrestoBlue reagent in differentiation media and allowed to incubate at 37° C. for 1 hour. Following incubation, supernatant was collected and analyzed for fluorescence intensity through excitation at 560 and emission at 590 to measure conversion of nonfluorescent resazurin to fluorescent resorufin via reduction by metabolic pathways in the cells. To conduct the luciferase assay, cell media collected and frozen throughout the study were thawed at room temperature. Samples from each well were assayed in duplicate by adding 5 μl of sample to 12.5 μl of Luciferase assay buffer. Coelenterazine reagent was then placed in each well and allowed to react for 10 minutes before reading the luminescence of each sample using a 10 millisecond integration time.

Results from the Presto Blue assay indicated similar viability between all fraction groups with the exception of the dexamethasone group having significantly lower viability compared to all groups and the aqueous high concentration group having significantly higher viability than the base cell culture media control. Dexamethasone is an inhibitor of muscle differentiation and this is reflected in the lower level of luciferase expression in cells treated with this media; the lower viability results from the myoblasts becoming less active in serum starvation if they are not allowed to differentiate.

Results from the luciferase assay indicated a significant increase in myoblast MLC expression within most of the fraction groups when compared to the media control and the dexamethasone supplemented media, negative control. The two exceptions to this were the low concentration of alginate and low concentration of aqueous which were only significantly greater than the dexamethasone group and not greater than the media control.

Thus, as shown by these experiments, the granule fraction of egg yolk had a positive effect on myoblast differentiation, thus indicative of a bioactive compound or multiple compounds within fertilized egg yolk improving lean muscle growth.

To identify specific bioactive compounds responsible for muscle growth contained in egg yolk, the granule portion was further subfractionated and subfractions were tested for their differentiation capabilities.

Granules are insoluble in aqueous solutions and form aggregates on the order of 1-8 um in size. In solutions containing 0.3 M NaCl, the particles begin to dissolve and become micelles on the order of 100-200 nm as the sodium disrupts the calcium bonding that causes the insoluble aggregates to form. Using phosphate buffered saline (PBS) as the solvent allowed for the granule fraction to dissolve from aggregate form and form micelles in solution. These micelles were then separated via centrifugal force and the biological effects of these subfractions in vitro within the reporter cell line were assessed. Additionally, the biological effects of unfertilized egg fractions compared to fertilized egg fractions was assessed.

Using centrifugation, three fractions were created that are composed of various size granules. The first fraction, the heaviest, referred to herein as F5, was isolated by centrifugation of solubilized granule at 5000 relative centrifugal force (rcf) for 15 minutes and is composed of the resuspended pellet obtained by this process. The next fraction, referred to herein as F7, was obtained by centrifugation of the supernatant from the previous step at 7500 rcf for 15 minutes and resuspension of the pellet. The last fraction with the lightest components, referred to herein as FS, was made up of the supernatant from the previous step.

All fractions were assayed via a bicinchoninic acid (BCA) assay to determine total protein content and run through a gel to validate their size ranges. For these analyses, ddH₂O at a level that produced a homogenous mixture was added. Samples were then taken from these mixtures and the aqueous fraction to conduct the BCA assay according to the manufacturer's instructions. Each sample was assayed at multiple concentrations 1×, 10×, 100× dilution. Samples were assayed in triplicate and allowed to react with the BCA buffer for 30 minutes at 37° C. Following the reaction, plates were read for absorbance at 562 nm and compared to a BSA standard controls of known concentration assayed at the same time to obtain an estimate of the protein concentration in each sample. Following the BCA assay, samples were run through a gel using SDS-PAGE to determine the content of each fraction. Samples were diluted to provide 5 μg of protein per lane when possible or the highest concentration available. The gel was then run at 200V for 35 minutes or until the dye reached the top reference line. Following the run, gels were fixed in methanol-acetone-acetic acid, and then stained with Coomassie blue for 24 hours. Following staining, gels were washed to remove excess stain using diH₂O and then imaged.

The myogenic effects of the F5, F7 and FS fractions on the reporter cell line disclosed herein were also examined.

Based upon these results, size exclusion chromatography was used to further separate proteins of the F7 and FS fractions into smaller subfractions. More specifically, the F7 and FS fractions were separated into subfractions based on their native conformations in PBS using FPLC on a Superdex 200 increase 10/300 column. Samples of one milliliter in volume were collected into microcentrifuge tubes and flash frozen and stored at −80° C. for later analysis and cell culture. Samples were named according to their elution volume and precursor fraction, for example F7.7 corresponds to the 7^{th} ml of liquid collected by running the F7 fraction through the column. Samples containing elutions with peaks in absorbance at 280 nm were then run on a gel. The F7 fractions showed diverse size ranges for most fractions and differing compositions for each fraction. For example, the F7.4 shows a band at 250 kDa and then bands at 75 kDa while the next observed fraction F7.6 had bands starting at 200 kDa and light bands at 75 kDa. The next fraction F7.7 once again has bands at 200 kDa. A gel for later elutions in the F7 subfraction showed lighter staining for most columns further down the gel indicating a trend of lighter proteins in those fractions. For the supernatant gels, even less correlation between size and fraction number was observed.

Samples collected from the chromatography were also measured for concentration using the BCA assay and the total protein concentration data was used to calculate the appropriate amount of each sample to add to the cell culture media to test each fraction's effect on differentiation when applied to myoblasts as described herein. Data from this assay are depicted in the Tables 1.1 and 1.2.

TABLE 1.1
Summary of statistical analysis for 7500 g fractions

| | |
|---|---|
| Luciferase | Viability |

| Group | 1% | Dex | IGF | 1% | Dex | IGF |
|---|---|---|---|---|---|---|
| F7.3L | + | + | n.s. | n.s. | + | − |
| F7.3H | + | + | n.s. | n.s. | + | − |
| F7.4L | n.s. | n.s. | n.s. | − | + | − |
| F7.4H | n.s. | n.s. | n.s. | n.s. | + | − |
| F7.6L | n.s. | n.s. | − | n.s. | + | − |
| F7.6H | n.s. | n.s. | − | n.s. | + | − |
| F7.7L | n.s. | n.s. | − | n.s. | + | − |
| F7.7H | − | n.s. | − | n.s. | + | − |
| F7.8L | − | − | − | n.s. | + | − |
| F7.8H | + | + | n.s. | n.s. | + | − |
| F7.11L | + | + | + | n.s. | + | − |
| F7.11H | + | + | + | + | + | − |
| F7.18L | + | + | + | + | + | − |
| F7.18H | + | + | + | + | + | − |
| F7.21L | + | + | + | + | + | − |
| F7.21H | + | + | + | + | + | − |

n.s. (non-significant); + (experimental group higher), − (experimental group lower; Tukey post hoc p < 0.05

TABLE 1.2
Summary of statistical analysis for Supernatant fractions

| | |
|---|---|
| Luciferase | Viability |

| Group | 1% | Dex | IGF | 1% | Dex | IGF |
|---|---|---|---|---|---|---|
| FS.3L | + | + | n.s. | + | + | − |
| FS.3H | + | + | n.s. | + | + | n.s. |
| FS.4L | + | + | n.s. | + | + | − |
| FS.4H | + | n.s. | n.s. | + | + | − |
| FS.7L | + | + | n.s. | n.s. | + | − |
| FS.7H | + | + | n.s. | n.s. | + | − |
| FS.10L | + | + | + | n.s. | + | − |
| FS.10H | + | + | + | n.s. | + | − |
| FS.11L | + | + | + | n.s. | + | − |
| FS.11H | + | + | + | n.s. | + | − |
| FS.17L | + | + | n.s. | + | + | n.s. |
| FS.17H | + | + | n.s. | + | + | n.s. |
| FS.22L | + | + | + | + | + | n.s. |
| FS.22H | + | + | + | + | + | n.s. |

n.s. (non-significant); + (experimental group higher), − (experimental group lower; Tukey post hoc p < 0.05

From these tables, several fractions were identified that induce MLC expression above that of the positive 1SF control at day 10; these were F7.11, F7.18, and F7.21 and FS.10, FS.11 and FS.22. These fractions, along with two control fractions F7.7 and FS.8 which do not exhibit myogenic activity, were then analyzed by mass spectrometry (MS) to identify compounds responsible for the observed myogenic activity. Each fraction was run through MS 3 times, for a total of 24 raw data files from the 8 fractions.

More specifically, samples were analyzed by LC-MS using Nano LC-MS/MS (Dionex Ultimate 3000 RLSCnano System, Thermofisher) interfaced with Eclipse (ThermoFisher). Three microliter out of 12.5 μl of in-gel digested sample was loaded on to a fused silica trap column (Acclaim PepMap 100, 75 um×2 cm, ThermoFisher). After washing for 5 minutes at 5 μl/minute with 0.1% TFA, the trap column was brought in-line with an analytical column (Nanoease MZ peptide BEH C18, 130A, 1.7 um, 75 um×250 mm, Waters) for LC-MS/MS. Peptides were fractionated at 300 nL/minute using a segmented linear gradient 4-15% Solution B in 30 minutes (where Solution A contains 0.2% formic acid, and Solution B contains 0.16% formic acid, 80% acetonitrile), 15-25% Solution B in 40 minutes, 25-50% Solution B in 44 minutes, and 50-90% Solution B in 11 minutes. Solution B was then returned at 4% for 5 minutes for the next run. The scan sequence began with an MS1 spectrum (Orbitrap analysis, resolution 120,000, scan range from M/Z 375-1500, automatic gain control (AGC) target 8E5, maximum injection time 100 ms). The top S (3 seconds) duty cycle scheme were used for determine number of MSMS performed for each cycle. Parent ions of charge 2-7 were selected for MSMS and dynamic exclusion of 60 seconds was used to avoid repeat sampling. Parent masses were isolated in the quadrupole with an isolation window of 1.2 m/z, automatic gain control (AGC) target 1E5, and fragmented with higher-energy collisional dissociation with a normalized collision energy of 30%. The fragments were scanned in Orbitrap with resolution of 15,000. The MSMS scan ranges were determined by the charge state of the parent ion but lower limit was set at 110 amu.

Proteins identified in initial analyses as being higher in concentration included albumin, ovalbumin, gelsolin, and lysozyme C. When the spectral count for each protein was divided by the total spectral counts in each sample to account for variation in sample concentration, some additional proteins that were different in terms of their expression when compared to the non-differentiating fractions were identified including: SMB domain-containing protein, transthyretin, IG-like domain-containing protein, fibrinogen C-terminal domain-containing protein, plasminogen, phosvitin (VTG2), peptidase S1 domain-containing protein, and fibrinogen C.

Further, using both spectral counting and intensive peptide identification search, 15 peptide sequences were identified in at least 5 of 6 active fractions, but not detected via this process in the 2 inactive fractions.

Table 2 shows these active peptides. Note by definition the 2 inactive fractions show 0.

TABLE 2

| Sample | | | | | | | F7.7 | FS.8 |
|---|---|---|---|---|---|---|---|---|
| Mpep | F7.11 | F7.18 | F7.21 | FS.10 | FS.11 | FS.22 | inactive | inactive |
| \hat{ }(42)LGES(80)ADLTAISVR$ | 4 | 1 | 0 | 1 | 5 | 2 | 0 | 0 |
| (SEQ ID NO: 1) | | | | | | | | |
| \hat{ }AVQLGLGPFPYC(57)R$ | 1 | 1 | 1 | 0 | 1 | 2 | 0 | 0 |
| (SEQ ID NO: 2) | | | | | | | | |
| \hat{ }DVVDLAQK$ | 1 | 1 | 2 | 5 | 2 | 0 | 0 | 0 |
| (SEQ ID NO: 3) | | | | | | | | |
| \hat{ }EPDNFWVALGGK$ | 2 | 1 | 0 | 1 | 1 | 1 | 0 | 0 |
| (SEQ ID NO: 4) | | | | | | | | |
| \hat{ }HTQVQVLPESGETPLFK$ | 1 | 1 | 0 | 2 | 2 | 2 | 0 | 0 |
| (SEQ ID NO: 5) | | | | | | | | |
| \hat{ }IEKVPFDAATLHTSR$ | 3 | 1 | 0 | 1 | 3 | 1 | 0 | 0 |
| (SEQ ID NO: 6) | | | | | | | | |
| \hat{ }LAAAVSNFGYDLYR$ | 1 | 1 | 1 | 0 | 2 | 2 | 0 | 0 |
| (SEQ ID NO: 7) | | | | | | | | |
| \hat{ }LLEGEESR$ | 1 | 3 | 2 | 2 | 1 | 3 | 0 | 0 |
| (SEQ ID NO: 8) | | | | | | | | |
| \hat{ }LQSLFTSPDFTK$ | 1 | 1 | 1 | 0 | 2 | 2 | 0 | 0 |
| (SEQ ID NO: 9) | | | | | | | | |
| \hat{ }LTPVAQELKELTK$ | 1 | 1 | 0 | 1 | 2 | 1 | 0 | 0 |
| (SEQ ID NO: 10) | | | | | | | | |
| \hat{ }NSDNDLVVNGIEIYR$ | 2 | 1 | 0 | 1 | 2 | 2 | 0 | 0 |
| (SEQ ID NO: 11) | | | | | | | | |
| \hat{ }QEY(80)DESGPSIVHR$ | 1 | 1 | 0 | 1 | 1 | 1 | 0 | 0 |
| (SEQ ID NO: 12) | | | | | | | | |
| \hat{ }VELQELNDR$ | 2 | 1 | 1 | 1 | 1 | 1 | 0 | 0 |
| (SEQ ID NO: 13) | | | | | | | | |
| \hat{ }VSAYIPWIK$ | 0 | 2 | 2 | 1 | 1 | 1 | 0 | 0 |
| (SEQ ID NO: 14) | | | | | | | | |
| \hat{ }VTVASWMR$ | 19 | 1 | 2 | 1 | 18 | 0 | 0 | 0 |
| (SEQ ID NO: 15) | | | | | | | | |

The symbols ‘\hat{ }’ and ‘$’ in the peptide sequences of Table 2 denote the n- and c-terminus, respectively. Numbers inside parentheses denote the mass of a presumed chemical modification to the preceding residue or terminus. For example, ‘\hat{ }(42)’ denotes acetylation of the n-terminus resulting in an additional roughly 42 amu mass, C(57) refers to (+57.021465) atomic mass units for carbamidomethyl modification to cysteine (C) and S(80), T(80) and Y(80) refer to (+79.96633) amu for phosphorylation to their amino acid residues.

Accordingly, provided herein are myogenic compounds and compositions thereof identified in separated fractions of fertilized avian egg yolk. As discussed supra, the myogenic compounds of this disclosure, and compositions thereof, are distinguishable from natural egg yolk in that the myogenic compounds are either physically separated by mechanical means from other components in natural egg yolk including, but not limited to, non-myogenic components of egg yolk and lipid components of egg yolk or produced by synthetic means, for example, via peptide synthesis or recombinant protein production by well-established protocols. Further, as will be understood by the skilled artisan upon reading this disclosure, while identified in avian egg yolk, other alternative biological sources are expected to contain the identified myogenic compounds and myogenic compounds of this disclosure may be derived from these alternative biological sources.

In one nonlimiting embodiment, the composition comprises one or more myogenic compounds identified in subfraction F7.11, F7.18, F7.21, FS.10, FS.11, and/or FS.22 of fertilized avian egg yolk or a synthetic derivative thereof.

By “subfraction F7.11”, it is meant a fraction obtained by resuspension of the pellet resulting from centrifugation at 7500 rcf for 15 minutes of a supernatant which resulted from centrifugation of solubilized egg yolk granule at 5000 rcf for 15 minutes (referred to herein as F7 fraction) which has been further subfractionated via size exclusion chromatography and corresponds to the 11^{th} ml of liquid collected by running the F7 fraction through the size exclusion column.

By “subfraction F7.18”, it is meant a fraction obtained by resuspension of the pellet resulting from centrifugation at 7500 rcf for 15 minutes of a supernatant which resulted from centrifugation of solubilized egg yolk granule at 5000 rcf for 15 minutes (referred to herein as F7 fraction) which has been further subfractionated via size exclusion chromatography and corresponds to the 18^{th} ml of liquid collected by running the F7 fraction through the size exclusion column.

By “subfraction F7.21”, it is meant a fraction obtained by resuspension of the pellet resulting from centrifugation at 7500 rcf for 15 minutes of a supernatant which resulted from centrifugation of solubilized egg yolk granule at 5000 rcf for 15 minutes (referred to herein as F7 fraction) which has been further subfractionated via size exclusion chromatography and corresponds to the 21^{st} ml of liquid collected by running the F7 fraction through the size exclusion column.

By “subfraction FS.10”, it is meant a fraction comprising the supernatant obtained from centrifugation at 7500 rcf for 15 minutes of a supernatant which resulted from centrifugation of solubilized egg yolk granule at 5000 rcf for 15 minutes (referred to herein as FS fraction) which has been further subfractionated via size exclusion chromatography and corresponds to the 10^{th} ml of liquid collected by running the FS fraction through the size exclusion column.

By “subfraction FS.11”, it is meant a fraction comprising the supernatant obtained from centrifugation at 7500 rcf for 15 minutes of a supernatant which resulted from centrifugation of solubilized egg yolk granule at 5000 rcf for 15 minutes (referred to herein as FS fraction) which has been further subfractionated via size exclusion chromatography and corresponds to the 11^{th} ml of liquid collected by running the FS fraction through the size exclusion column.

By “subfraction FS.22”, it is meant a fraction comprising the supernatant obtained from centrifugation at 7500 rcf for 15 minutes of a supernatant which resulted from centrifugation of solubilized egg yolk granule at 5000 rcf for 15 minutes (referred to herein as FS fraction) which has been further subfractionated via size exclusion chromatography and corresponds to the 21^{st}/22^{nd} ml of liquid collected by running the FS fraction through the size exclusion column.

In one nonlimiting embodiment, the composition comprises a powder prepared from drying one or more of subfractions F7.11, F7.18, F7.21, FS.10, FS.11, and/or FS.22.

In one nonlimiting embodiment, the composition comprises one or more myogenic compounds comprising one or more peptide sequences depicted in Table 2. In one nonlimiting embodiment, the myogenic compound is a protein comprising a peptide sequence depicted in Table 2 or a myogenically active fragment thereof. In one nonlimiting embodiment, the myogenic compound is a protein selected from gelsolin, actin-depolymerizing factor, vimentin, SERPIN domain-containing protein, also referred to as pigment epithelium derived factor (PEDF), hepatocyte growth factor activator, chick nucleoside diphosphate kinase, inter-alpha-trypsin inhibitor heavy chain, keratin-type II cytoskeletal cochleal, desmin, apolipoprotein A-I, albumin, actin-cytoplasmic type 5, actin-cytoplasmic 1, vitellogenin-1, actin-cytoplasmic 2, IF rod domain-containing protein, vitellogenin-3, glial fibrillary acidic protein, plasminogen or type II alpha-keratin IIA or a myogenically active peptide fragment thereof.

In one nonlimiting embodiment, the myogenic compound is a protein selected from albumin, ovalbumin, gelsolin, lysozyme C, SMB domain-containing protein, transthyretin, IG-like domain-containing protein, fibrinogen C-terminal domain-containing protein, plasminogen, phosvitin (VTG2), peptidase Si domain-containing protein or fibrinogen C or a myogenically active fragment thereof.

In one nonlimiting embodiment, the myogenic compound is a protein which exhibits at least 70%, 80%, 90%, 95% or 99% sequence identity to a peptide sequence or protein identified herein and exhibits myogenic activity.

In one nonlimiting embodiment, the composition comprises one or more synthetic derivatives of a myogenic compound identified in subfraction F7.11, F7.18, F7.21, FS.10, FS.11, and/or FS.22 of fertilized avian egg yolk or comprising one or more peptide sequences depicted in Table 2. In one embodiment, the synthetic derivative is prepared by recombinant protein expression using a well-established method. In one nonlimiting embodiment, the synthetic derivative is a recombinant protein or myogenically active fragment thereof comprising a peptide sequence depicted in Table 2. In one nonlimiting embodiment, the synthetic derivative is a recombinant protein or myogenically active fragment thereof which exhibits at least 70%, 80%, 90%, 95% or 99% sequence identity to a peptide sequence or protein identified herein and exhibits myogenic activity.

By “myogenically active fragment” or “myogenically active peptide fragment” as used herein, it meant a peptide sequence shorter in amino acid sequence than a full length protein but which maintains similar myogenic activity to the full length protein.

In one nonlimiting embodiment, the myogenic compound is a protein or myogenically active peptide fragment thereof similar to that identified herein but is from a species alternative to chick such as, but not limited to, humans, dogs, cats, horses, cows, sheep, pigs and primates.

In one nonlimiting embodiment, the myogenic compound is a recombinant human protein or myogenically active peptide fragment thereof.

The myogenic effects of several individual compounds identified herein, namely chick SERPIN domain-containing protein, also referred to as pigment epithelium derived factor (PEDF) and a chick nucleoside diphosphate kinase as well as human recombinant proteins thereof, SERPINF1 and a human recombinant nucleoside diphosphate kinase referred to as NME2, as well as FORTETROPIN, on proliferation of murine C2C12 cells and activation of MLC1f promoter by secreted Gaussia Luciferase were examined.

The chick SERPIN domain-containing protein obtained from an ELISA kit significantly increased GLuc activity by 124-145% in the C2C12 cell line after four days of treatment. The vehicle of the chick nucleoside diphosphate kinase protein from an ELISA kit was toxic to the C2C12 cells so myogenic activity of this protein could not be assessed with the materials on hand. However, both human recombinant proteins thereof, SERPINF1 and the human recombinant nucleoside diphosphate kinase referred to as NME2 stimulated statistically significant proliferation of murine C2C12 cell line at concentrations ranging from 62.5 to 1000 ng/ml from 109% to 120% of control for SERPINF1 and 107% to 118% of control for NME2, respectively. Further, human recombinant SERPINF1 protein statistically significantly activated the MLC1f promoter determined via secreted GLuc in a dose-dependent manner in the C2C12 cell line after three days of treatment (from 128% to 115% with 250 ng/ml and 125 ng/ml respectively) and on day seven of treatment (from 141% to 135% with 250 ng/ml to 15.6 ng/ml). Human recombinant NME2 protein also statistically significantly activated MLC1f promoter determined via secreted GLuc in a dose-dependent manner in the C2C12 cell line after three days of treatment (from 135.8% to 121.2% with 250 ng/ml and 125 ng/ml respectively) and on day seven of treatment (from 126.4% to 113% with 250 ng/ml to 15.6 ng/ml). Protein extracts from FORTETROPIN at various pH levels and different incubation times also significantly increased GLuc activity (133-162%) in C2C12 cell line following 4 days after treatment.

Further, the presence of the chick SERPIN domain-containing protein, also referred to as pigment epithelium derived factor (PEDF) and the chick nucleoside diphosphate kinase in the myogenically active fertilized egg yolk product, FORTETROPIN was confirmed via ELISA assays with the highest concentration of chick SERPIN domain-containing protein extracted from one gram of FORTETROPIN being 140 ng and the highest concentration of chick nucleoside diphosphate kinase extracted from one gram of FORTETROPIN being 1270 ng.

The myogenic compounds identified herein can be formulated into nutraceutical and/or pharmaceutical compositions for use in increasing muscle mass in a mammal. In one nonlimiting embodiment, the nutraceutical and/or pharmaceutical composition comprises more than one of the myogenic compounds identified herein. In one nonlimiting embodiment, the nutraceutical and/or pharmaceutical composition further comprises powdered egg yolk. In one nonlimiting embodiment, the nutraceutical and/or pharmaceutical composition further comprises FORTETROPIN. FORTETROPIN is a fertilized egg yolk derived product used as a dietary and nutritional supplement (MYOS CORP., Cedar Knolls, NJ). A method for production of FORTETROPIN is disclosed in U.S. Pat. No. 8,815,320, teachings of which are herein incorporated by reference in their entirety. In another embodiment, the nutraceutical and/or pharmaceutical composition further comprises a spray dried egg yolk powder such as described in U.S. patent application Ser. No. 16/151,601, the disclosure of which is incorporated herein by reference in its entirety. In one nonlimiting embodiment, the nutraceutical and/or pharmaceutical composition increases muscle growth in a mammal similarly to powdered egg yolk. In one nonlimiting embodiment, the nutraceutical and/or pharmaceutical composition increases muscle growth to a greater extent as compared to powdered egg yolk.

In compositions comprising more than one myogenic compound as disclosed herein, compounds may be additive in myogenic activity or synergistic in myogenic activity, meaning more than an additive effect.

As will be understood by the skilled artisan upon reading this disclosure, the compositions described herein can be formulated for administration to a mammal via any conventional means including, but not limited to, oral, or buccal.

Moreover, the compositions described herein, can be formulated into any suitable dosage form, including but not limited to, aqueous oral dispersions, liquids, gels, syrups, elixirs, slurries, suspensions and the like, for oral ingestion by an individual in need, solid oral dosage forms, controlled release formulations, fast melt formulations, effervescent formulations, lyophilized formulations, tablets, powders, pills, dragees, capsules, delayed release formulations, aqueous liquid dispersions, self-emulsifying dispersions, solid solutions, liposomal dispersions, solid dosage forms, powders, tablets, capsules, pills, delayed release formulations.

Formulations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including glucose, fructose, lactose, sucrose, mannitol, sorbitol, stevia extract, or sucralose; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Formulations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, pellets, granules. In other embodiments, the pharmaceutical formulation is in the form of a powder. In still other embodiments, the pharmaceutical formulation is in the form of a tablet. Additionally, formulations described herein may be administered as a single capsule or in multiple capsule dosage form. In some embodiments, the formulation is administered in two, or three, or four, capsules or tablets.

Soft gel or soft gelatin capsules may be prepared, for example, without limitation, by dispersing the formulation in an appropriate vehicle (vegetable oils are commonly used) to form a high viscosity mixture. This mixture is then encapsulated with a gelatin-based film using technology and machinery known to those in the soft gel industry. The industrial units so formed are then dried to constant weight.

In some embodiments, the formulations may include other medicinal or pharmaceutical agents, carriers, diluents, dispersing agents, suspending agents, thickening agents, adjuvants, such as preserving, stabilizing, wetting or emulsifying agents, solution promoters, and/or buffers. In addition, the formulations can also contain other therapeutically valuable substances.

The formulations described herein can include one or more myogenic compounds and one or more nutraceutically or pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination(s) thereof. In still other aspects, using standard coating procedures, a film coating is provided around the formulation of the compound described herein. In one embodiment, some or all of the particles of the compound described herein are coated. In another embodiment, some or all of the particles of the compound described herein are microencapsulated. In still another embodiment, the particles of the compound described herein are not microencapsulated and are uncoated.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Formulations including one or more myogenic compounds, as described herein, may be manufactured in a conventional manner, such as, by way of example only, by means of conventional mixing, dissolving, granulating, dragee-making, levigating, emulsifying, encapsulating, entrapping or compression processes.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

Binders imparting cohesive qualities may also be used. Examples include, but are not limited to, alginic acid and salts thereof; cellulose derivatives such as carboxymethylcellulose, methylcellulose, hydroxypropylmethylcellulose, hydroxyethylcellulose, hydroxypropylcellulose, ethylcellulose, and microcrystalline cellulose; microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose, glucose, dextrose, molasses, mannitol, sorbitol, xylitol, and lactose; a natural or synthetic gum such as acacia, tragacanth, ghatti gum, mucilage of isapol husks, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder.

Formulators skilled in art can determine the binder level for the formulations, but binder usage level of up to 70% in tablet formulations is common.

Compositions may further comprise carriers of relatively nontoxic chemical compounds or agents that facilitate the incorporation of a compound into cells or tissues. Nonlimiting examples include binders, suspending agents, disintegration agents, filling agents, surfactants, solubilizers, stabilizers, lubricants, wetting agents, diluents, and the like. Suitable carriers for use in solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Dispersing agents and/or viscosity modulating agents include materials that control the diffusion and homogeneity of a compound through liquid media or a granulation method or blend method. In some embodiments, these agents also facilitate the effectiveness of a coating or eroding matrix. Nonlimiting examples of diffusion facilitators/dispersing agents include hydrophilic polymers, electrolytes, a Tween, PEG, polyvinylpyrrolidone, and carbohydrate-based dispersing agents such as hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers, block copolymers of ethylene oxide and propylene oxide; and poloxamines, tetrafunctional block copolymers derived from sequential addition of propylene oxide and ethylene oxide to ethylenediamine, polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents that are particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

Compositions of the present invention may further comprise diluents used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose; dibasic calcium phosphate, dicalcium phosphate dihydrate; tricalcium phosphate, calcium phosphate; anhydrous lactose, spray-dried lactose; pregelatinized starch, compressible sugar; mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; sodium chloride; inositol, bentonite, and the like.

Compositions may further comprise an enteric coating, a substance that remains substantially intact in the stomach but dissolves and releases the myogenic compound in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

In addition, the compositions may comprise an erosion facilitator, a material that controls the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

Filling agents including compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like can also be included in the compositions. Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In addition, flavoring agents and/or sweeteners can be used in the compositions and may include acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate, maltol, mannitol, maple, marshmallow, menthol, mint cream, mixed berry, neohesperidine DC, neotame, orange, pear, peach, peppermint, peppermint cream, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

The compositions may further comprise lubricants and/or glidants that prevent, reduce or inhibit adhesion or friction of materials. Nonlimiting examples of lubricants include stearic acid, calcium hydroxide, talc, sodium stearyl fumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG-4000) or a methoxypolyethylene glycol, sodium oleate, sodium benzoate, glyceryl behenate, polyethylene glycol, magnesium or sodium lauryl sulfate, colloidal silica, a starch such as corn starch, silicone oil, a surfactant, and the like.

Plasticizers, compounds used to soften the microencapsulation material or film coatings to make them less brittle may also be included in the compositions. Examples of suitable plasticizers include, but are not limited to, polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

The compositions may further comprise solubilizers such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

In addition, the compositions my comprise stabilizers such as antioxidation agents, buffers, acids, preservatives and the like.

Suitable suspending agents for use in solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants including compounds such as sodium lauryl sulfate, sodium docusate, Tweens, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide and the like may also be included. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

Viscosity enhancing agents including, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof may also be included.

In addition, wetting agents including compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like may be included in these compositions.

In some embodiments, solid dosage forms, e.g., tablets, capsules, are prepared by mixing the myogenic compound described herein, with one or more pharmaceutical excipients to form a bulk blend composition. When referring to these bulk blend compositions as homogeneous, it is meant that the particles of myogenic compound are dispersed evenly throughout the composition so that the composition may be readily subdivided into equally effective unit dosage forms, such as tablets, pills, and capsules.

Conventional techniques include, e.g., one or a combination of methods: (1) dry mixing, (2) direct compression, (3) milling, (4) dry or non-aqueous granulation, (5) wet granulation, or (6) fusion. See, e.g., Lachman et al., “The Theory and Practice of Industrial Pharmacy” (1986).

It should be appreciated that there is considerable overlap between additives used in the solid dosage forms described herein. Thus, the above-listed additives should be taken as merely exemplary, and not limiting, of the types of additives that can be included.

A capsule may be prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials which sufficiently isolate the compound from other non-compatible excipients. Materials compatible with the myogenic compound are those that delay the release of the myogenic compound in vivo.

In other embodiments, the formulations described herein, which include the myogenic compound, are solid dispersions. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl 2004/0013734.

In still other embodiments, the formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions are known in the art and include, but are not limited to, for example, U.S. Pat. Nos. 4,151,273, 5,281,420, and 6,083,518.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components. The enteric coated oral dosage form may also be a capsule (coated or uncoated) containing pellets, beads or granules of the solid carrier or the composition.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating for the methods and compositions described herein to achieve delivery to the lower gastrointestinal tract.

In some embodiments, formulations are provided that include particles of myogenic compound described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of myogenic compound, the liquid dosage forms may include additives, such as: (a) disintegrating agents; (b) dispersing agents; (c) wetting agents; (d) at least one preservative, (e) viscosity enhancing agents, (t) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regards to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In one nonlimiting embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the formulations described herein can be self-emulsifying drug delivery systems (SEDDS). Emulsions are dispersions of one immiscible phase in another, usually in the form of droplets. Generally, emulsions are created by vigorous mechanical dispersion.

SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563.

Buccal formulations that include myogenic compound may be administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with the myogenic compound, and any other components that may be present in the buccal dosage unit. Generally, the polymeric carrier comprises hydrophilic (water-soluble and water-swellable) polymers that adhere to the wet surface of the buccal mucosa. Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

In certain embodiments, delivery systems for pharmaceutical compounds may be employed, such as, for example, liposomes and emulsions. In certain embodiments, compositions provided herein can also include a mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In one nonlimiting embodiment, the nutraceutical and/or pharmaceutical composition may further comprise powdered egg yolk. In one nonlimiting embodiment, the nutraceutical and/or pharmaceutical composition may further comprise FORTETROPIN.

The following nonlimiting examples further illustrate the present invention.

### EXAMPLES

### Example 1: Evaluation of the Effect of Chick SERPINF1, Chick NME2 and Proteins Extracted from FORTETROPIN on Activation of MLC1f Promoter by Secreted Gaussia Luciferase

### Materials and Methods

### Compounds

Compounds tested included FORTETROPIN provided by MYOS Corp.; Chicken Nucleoside Diphosphate Kinase B (NME2) from ELISA kit standards (cat #MBS7244605; MyBioSource); chicken SERPINF1 or Chicken Pigment Epithelium Derived Factor (PEDF) standard from ELISA kit (cat #MBS264953; MyBioSource); recombinant murine IGF1 from Peprotech; and dexamethasone from TCI America

### Cell Culture

For cell culture, a C2C12 mouse muscle myoblast cell line carrying a reporter gene Gaussia Luciferase (GLuc) under the muscle specific promoter for fast myosin light chain MLCf1 was used. Cells were expanded in proliferation media consisting of DMEM 4.5 g/L supplemented with 10% v/v fetal bovine serum, and 1 U/ml penicillin streptomycin. The C2C12 myoblast cell line was seeded in 48-well plates at a density of 25,000 cells/cm² and cultured in proliferation media, for the first 3 days changing media once per day. Three wells per each condition. On day 3 the media was changed to differentiation media consisting of DMEM 4.5 g/L glucose supplemented with 1% FBS, 1 U/ml penicillin streptomycin. To confirm that the experimental conditions were appropriate and that the experiment was successful the activation of the reporter gene GLuc in the cell line was tested under three different treatment conditions: vehicle/media control; positive control containing 50 ng/ml of insulin-like growth factor (IGF1); and negative control containing 50 μg/ml of dexamethasone.

Protein Extraction from FORTETROPIN Powder

Protein extraction was performed following an established protocol of Chalamaiah et al. (Food Chem 2018 268:369-377) to ensure accurate and reliable results. Briefly, 0.2 or 2 grams of powdered FORTETROPIN were carefully reconstituted in 10 ml of phosphate buffered saline at varying pH levels of 4, 7, and 10. The mixtures were then incubated at room temperature for 3 hours or left for 48 hours. During the 3 hour incubation periodic vortexing was done every 30 minutes to promote effective extraction.

Once the incubation period was completed, the solutions were carefully centrifuged at 2000 RPM and transferred to Eppendorf tubes. For the further use of extracted proteins, the pH of the solutions was neutralized to pH7 before subjecting them to a second centrifugation step at 15,000 RPM. The extracted proteins were subsequently utilized for ELISA analysis, to determine the concentration of SERPINF1 and NME2.

The FORTETROPIN extracts were also used in cytotoxicity assays and activation of MLC1f promoter reporter gene assays, which allowed for a comprehensive evaluation of their biological activities.

### ELISA

ELISA assay was performed according to manufacturer's protocols to determine concentration of chicken SERPINF1/Pigment Epithelium Derived Factor (PEDF) and chicken nucleoside diphosphate kinase B (NME2) in the FORTETROPIN extracts.

ELISA Kits used were Chicken Nucleoside Diphosphate Kinase B (NME2) ELISA kit (cat #MBS7244605; MyBioSource) and Chicken Pigment Epithelium Derived Factor (PEDF) ELISA kit (cat #MBS264953; MyBioSource).

### Cytotoxicity Assay

The evaluation of cytotoxicity is a crucial step in drug development. Cytotoxicity refers to a drug's ability to cause damage or death to cells. Without evaluating cytotoxicity, using high concentrations of a drug in testing can kill cells, making it difficult to determine whether the drug is toxic or has an anti-proliferative effect. Cytotoxicity was evaluated using the Cell Titer96 Aqueous One (Promega, WI, USA) basic test according to manufacturer's instructions. Cells were seeded at a density of 10,000 cells/well in a 96-well plate and cultured overnight. Treatments were carried out for 24 hours with different doses of compounds, in triplicate. Colorimetric analysis was performed using an absorbance of 490 nm. Inhibition of viability of more than 20% of the control values was considered cytotoxic.

### Evaluation of Activation of MLC1f Promoter by Secreted Gaussia Luciferase (GLuc)

To evaluate activation of MLCf1 promoter by secreted Gluc, the MLC1f reporter C2C12 myoblasts cells were initially seeded in 48-well plates at a density of 25,000 cells/cm² and cultured in proliferation media (DMEM 4.5 g/L glucose with 10% FBS and 1 u/ml penicillin streptomycin) the first 3 days, with daily media changes at 37° C. in 5% CO₂ in a cell culture incubator.

On day 3, the media was switched to differentiation media comprising DMEM 4.5 g/L glucose with 1% FBS and 1 u/ml penicillin streptomycin. Protein extracts from 2% and 20% FORTETROPIN under different pH and the SERPINF1 protein standard from ELISA kit were evaluated.

Moreover, three control groups (n=3) were included in the study: 1) cells supplemented with 50 ng/mL murine IGF1 (Peprotech) to promote myoblast differentiation, 2) cells supplemented with 50 μg/mL dexamethasone to discourage myoblast differentiation, and 3) cells fed with the base differentiation media without any additives for the vehicle control.

Following 1-day or 4-days and 7 days of incubation, the supernatant from cells was collected, and stored at −80° C. GLuc assay was performed according to manufacturer's protocol (Thermo Fisher Scientific; Pierce Gaussia Luciferase Glow Assay Kit cat #16161).

### Statistics

GraphPad Prism 6.05 software (GraphPad Software, La Jolla, CA, USA) was used to calculate mean±standard deviation for each group and to perform statistical analysis using the One-Way ANOVA test with Dunnett's multiple comparisons post-test. *P<0.05 and **P<0.01.

### Results

### Cytotoxicity Analysis

Dexamethasone at concentrations from up to 100 μg/ml to 12.5 μg/ml showed no cytotoxicity on cultured C2C12 cell line.

An 88.3% to 80% cytotoxic effect was observed when using diluted NME2 proteins from the ELISA kit. However, even at 0 concentration of proteins from the ELISA kit, there was still toxicity, suggesting the presence of proprietary supplements required for ELISA, and not the NME2 protein were harmful to the cell culture.

SERPINF1/Pigment Epithelium Derived Factor (PEDF) lyophilized standard from ELISA kit is only cytotoxic at concentrations of 10 ng/ml.

Proteins extracted from FORTETROPIN at different pHs were cytotoxic at undiluted and 1:2 dilution but not at 1:4 dilution.

The cytotoxicity experiments were used to identify the highest non-toxic concentrations of the tested substances.

These concentrations were then chosen for further assessment of their biological activity in the C2C12 cell line.

Evaluation of NME2 and SERPINF1 Protein Concentration Using ELISA, after Protein Extraction from FORTETROPIN Powder Under Various pHs and Incubation Times

Based on ELISA data the maximum NME2 was extracted after a 48 hour protein extraction at pH 10 from 2% FORTETROPIN at room temperature. It is estimated that 1 gram of FORTETROPIN contains approximately 1,270 ng NME2.

Based on ELISA data the maximum SERPINF1 was also extracted after a 48 hour protein extraction at pH=10 from 2% FORTETROPIN at room temperature. It is estimated that 1 gram of FORTETROPIN contains approximately 140 ng SERPINF1.

Evaluation of the Effect of SERPINF1 and Proteins Extracted from FORTETROPIN on Activation of MLC1f Promoter by Secreted Gaussia Luciferase

The activation of the MLC1f promoter was assessed by different treatments using the measurement of secreted Gaussia Luciferase. Monitoring the levels of secreted Gaussia Luciferase allows for quantification of the promoter's activity under various experimental conditions. C2C12 cell lines were cultured in a 48-well plate for 5 days, with daily medium changes. First two days medium was replaced with growth medium. Last 3 days, the medium was replaced with differentiation medium. On day 5 of the experiment, IGF1 at 50 ng/ml was used as a positive control, dexamethasone at 50 μg/ml was used as a negative control, and medium was used as a vehicle control. Supernatant samples were collected for G-luciferase assay on day 3, day 4, and day 7.

The luciferase activity was directly assessed and compared as a measure of promoter activity without protein concentration normalization.

On day one, no induction of luciferase activity was observed, even with the positive control IGF1.

On day 4 post treatment, a significant activation of the MLC1f promoter-driven luciferase by 144.6±5.1 was observed in response to the positive control IGF1.

Meanwhile, the vehicle control and negative control dexamethasone had no effect, showing luciferase activities of 106.5±12.4 and 102±3.4, respectively. These results demonstrate that the experimental settings are correct, and the experiment is functioning as expected, validating the reliability of our findings.

SERPINF1 protein from the ELISA kit significantly increased GLuc activity by 124-145% in the C2C12 cell line after four days of treatment. There was a reverse dose-dependent activation of the Gluc reporter, suggesting interference from proprietary compounds in the ELISA kit. Dilution of the protein showed increased activity, indicating reduced interference at lower concentrations.

Protein extracts of FORTETROPIN at various pH levels and different incubation time significantly increase GLuc activity (133-162%) in C2C12 cell line following 4 days after treatment.

On day 7, there was a general decrease in luciferase activity across most treated samples. However, in the positive control IGF1 treated cells, even at low concentrations of SERPINF1 and extracted protein from FORTETROPIN at pH 10, statistically significant differences persisted between the treated and untreated cells.

### Example 2: Evaluation of the Effect of Human Recombinant Proteins SERPINF1 and NME2 on Proliferation of Murine C2C12 Cells and Activation of MLC1f Promoter by Secreted Gaussia Luciferase

### Materials and Methods

### Compounds Tested

NME2 human recombinant protein was obtained from MyBioSource (cat #206177). Human Pigment Epithelium-Derived Factor (PEDF) is a secreted glycoprotein encoded by the SERPINF1 gene and was also obtained from MyBioSource (cat #143337). Recombinant murine IGF1 was obtained from Peprotech and dexamethasone was obtained from TCI America.

### Cell Culture, Cytotoxicity Assay, Evaluation of Activation of MLC1f Promoter by Secreted Gaussia Luciferase (GLuc) and Statistics

Cell culture, cytotoxicity assay, evaluation of activation of MLC1f promoter by secreted GLuc and statistics were performed as described in Example 1.

### Results

### Cytotoxicity Analysis

Dexamethasone at concentrations from up to 100 μg/ml to 12.5 μg/ml showed no cytotoxicity on cultured C2C12 cell line. Further, human SERPINF1/Pigment Epithelium Derived Factor (PEDF) human recombinant NME2 protein and showed no cytotoxicity.

Human recombinant SERPINF1 stimulated statistically significant proliferation of murine C2C12 cell line at concentration starting from 1000 ng/ml to 62.5 ng/ml from 120% to 109% of control respectively.

Human recombinant NME2 stimulated statistically significant proliferation of murine C2C12 cell line at concentrations starting from 1000 ng/ml to 62.5 ng/ml from 118% to 107% of control respectively.

After conducting cytotoxicity experiments, the highest non-toxic concentrations of the tested substances were identified and these concentrations were then chosen for further assessment of their biological activity in the C2C12 cell line.

### Evaluation of the Effect of Recombinant Human SERPINF1 and NME2 Proteins, on Activation of MLC1f Promoter by Secreted Gaussia Luciferase.

The activation of the MLC1f promoter by different treatments was assessed using the measurement of secreted Gaussia Luciferase (GLuc). By monitoring the levels of secreted GLuc, the promoter's activity was quantified under various experimental conditions.

C2C12 cell lines were cultured in a 48-well plate for 5 days, with daily medium changes. First two days medium was replaced with growth medium. Last 3 days, the medium was replaced with differentiation medium. On day 5 of the experiment, IGF1 at 50 ng/ml was used as a positive control, dexamethasone at 25 μg/ml was used as a negative control, and medium was used as a vehicle control. Supernatant samples were collected for GLuc assay on day 1, day 4, and day 7. Luciferase activity was directly assessed and compared as a measure of promoter activity without protein concentration normalization.

On day one, no induction of luciferase activity was observed even with the positive control IGF1.

On day 4 post treatment, significant activation of the MLC1f promoter-driven luciferase by 172.4±4.8 in response to the positive control IGF1 was observed. Meanwhile, the negative control dexamethasone had no effect, showing luciferase activities of 100.4±1.4. These results demonstrate that the experimental settings are correct, and the experiment is functioning as expected, validating the reliability of our findings.

Treatment of murine C2C12 cell line with different concentrations of human recombinant SERPINF1 protein activated the MLC1f promoter is a dose-dependent manner. Specifically, human recombinant SERPINF1 protein statistically significantly activated MLC1f promoter as determined via secreted GLuc in the C2C12 cell line after three days of treatment (from 115% to 128% with 125 ng/ml and 250 ng/ml respectively) and on day seven of treatment (from 135% to 141% with 15.6 ng/ml to 250 ng/ml).

Importantly, on day 7 there was a decrease in luciferase activity in positive control IGF1 (from 172% on day 3 to 162% on day 7) while luciferase activity cells treated with SERPINF1 was going up (in 250 ng/ml treated samples the activity went from 128% on day 3 to 140% on day 7).

Similar to the treatment of murine C2C12 cell line with different concentrations of human recombinant SERPINF1 protein, dose-dependent activation of the MLC1f promoter was also observed with NME2 treatment. Specifically, human recombinant NME2 protein statistically significantly activated MLC1f promoter as determined via secreted GLuc in the C2C12 cell line after three days of treatment (from 121.2% to 135.8% with 125 ng/ml and 250 ng/ml respectively) and on day seven of treatment (from 113% to 126.4% with 15.6 ng/ml to 250 ng/ml). On day 7 when there was a decrease in luciferase activity in positive control IGF1 (from 172% on day 3 to 162% on day 7), cells treated with NME2 still showed increasing activity.

1: A composition comprising one or more myogenic compounds identified in a fraction of egg yolk or a synthetic derivative thereof.

2: The composition of claim 1 wherein the egg yolk is avian egg yolk.

3: The composition of claim 2 wherein the egg yolk is fertilized avian egg yolk.

4: The composition of claim 3 wherein the one or more myogenic compounds is identified in subfraction F7.11, F7.18, F7.21, FS.10, FS.11, and/or FS.22 of fertilized avian egg yolk.

5: The composition of claim 3 wherein the one or myogenic compounds is a synthetic derivative of a myogenic compound isolated from subfraction F7.11, F7.18, F7.21, FS.10, FS.11, and/or FS.22 of fertilized avian egg yolk.

6: The composition of claim 1 wherein the one or more myogenic compounds comprises a peptide sequence depicted in Table 2 or a peptide sequence with at least 70% sequence identity to a peptide sequence depicted in Table 2.

7: The composition of claim 1 wherein the myogenic compound is a protein or myogenically active fragment thereof.

8: The composition of claim 7 wherein the protein is selected from gelsolin, actin-depolymerizing factor, vimentin, SERPIN domain-containing protein, pigment epithelium derived factor, chick nucleoside diphosphate kinase, hepatocyte growth factor activator, inter-alpha-trypsin inhibitor heavy chain, keratin-type II cytoskeletal cochleal, desmin, apolipoprotein A-I, albumin, actin-cytoplasmic type 5, actin-cytoplasmic 1, vitellogenin-1, actin-cytoplasmic 2, IF rod domain-containing protein, vitellogenin-3, glial fibrillary acidic protein, plasminogen and/or type II alpha-keratin IIA or a myogenically active fragment thereof.

9: The composition of claim 7 wherein the protein is selected from albumin, ovalbumin, gelsolin, lysozyme C, SMB domain-containing protein, transthyretin, IG-like domain-containing protein, fibrinogen C-terminal domain-containing protein, plasminogen, phosvitin (VTG2), peptidase S1 domain-containing protein, and/or fibrinogen C or a myogenically active fragment thereof.

10: The composition of claim 1 wherein the synthetic derivative is from a species alternative to chick.

11: The composition of claim 10 wherein the species alternative to chick is selected from human, dog, cat, horse, cow, sheep, pig or primate.

12: A nutraceutical composition comprising the composition of claim 1 and a nutraceutically acceptable excipient.

13: A pharmaceutical composition comprising the composition of claim 1 and a pharmaceutically acceptable excipient.

14: The nutraceutical composition of claim 12 further comprising powdered egg yolk.

15: The nutraceutical composition of claim 12 further comprising FORTETROPIN.

16: A method for increasing muscle mass in a mammal, said method comprising administering to the mammal a composition of claim 1.

17: The pharmaceutical composition of claim 13 further comprising powdered egg yolk.

18: The pharmaceutical composition of claim 13 further comprising FORTETROPIN.

19: A method for increasing muscle mass in a mammal, said method comprising administering to the mammal a nutraceutical composition of claim 12.

20: A method for increasing muscle mass in a mammal, said method comprising administering to the mammal a pharmaceutical composition of claim 13.
